(19) United States
(12) Patent Application Publication
(10) Pub. No.: US 2022/0033291 A1
(43) Pub. Date: Feb. 3, 2022

(54) HIGH RATE ACIDIFICATION AND ORGANIC SOLIDS SOLUBILIZATION PROCESS

(71) Applicant: GREENFIELD GLOBAL INC., Toronto (CA)

(72) Inventors: Hisham Mohamed HAFEZ, London (CA); Ashar SAYEED, Chatham (CA); Christopher Bruce BRADT, La Salle (CA); David Alan SALT, Mississauga (CA)

(21) Appl. No.: 17/276,933

(22) PCT Filed: Sep. 17, 2019
(86) PCT No.: PCT/CA2019/051318

§ 371 Date: Mar. 17, 2021

Related U.S. Application Data
(60) Provisional application No. 62/732,695, filed on Sep. 18, 2018.

(51) Int. Cl. C02F 9/00; C12P 3/00

(52) U.S. Cl. CPC C02F 9/00; C02F 1/66; C12P 3/00

(57) ABSTRACT

A method and system for high rate acidification and organic solids solubilization of feedstocks such as municipal source separated organics, municipal sewage sludge, and various industrial organic wastes are disclosed. The method and system feature a completely mixed bioreactor containing hydrogen-producing microorganisms, a crossflow membrane unit or membrane module located downstream of the bioreactor, a storage tank for receiving concentrated microorganisms from the membrane unit or module, and a connection that recirculates desired quantities of biomass from the storage tank to the bioreactor. This configuration decouples the solids residence time (SRT) from the hydraulic retention time (HRT) and results in a high solubilization rate.

### CROSS REFERENCE TO RELATED APPLICATION

This application claims priority to U.S. provisional patent application U.S. 62/732,695, filed Sep. 18, 2018, the entire contents of which is hereby incorporated by reference.

### FIELD

The present disclosure relates generally to a method and system for high rate acidification and organic solids solubilization of feed stocks such as thin stillage from a corn-based ethanol plant, municipal source separated organics, municipal sewage sludge, and various industrial organic wastes.

### BACKGROUND

The continuously stirred tank reactor (CSTR) has been the most widely used system for continuous hydrogen production (Li and Fang, 2007). Since in a CSTR biomass solids residence time (SRT) is the same as the hydraulic retention time (HRT), its concentration in the mixed liquor is highly affected by the recommended HRT of 1-12 h which is optimal for high hydrogen production rates (Li and Fang, 2007). The maximum specific growth rate (pmax) for mixed culture of 0.333 h⁻¹ (Horiuchi et al., 2002) corresponds to a SRTmin of 3.0 h.

However, high dilution rates result in a marked decrease in biomass content in the reactor due to severe cell washout and system failure (Wu et al., 2008). Decoupling of SRT from HRT in hydrogen bioreactors has been achieved primarily by using biofilms on several media including synthetic plastic media and treated anaerobic granular sludge (Das et al., 2008), activated carbon, expanded clay and loofah sponge (Chang et al., 2002), and glass beads (Zhang et al., 2006). Problems with the development of methanogenic bacteria in the acidification process adversely impact process stability, which is critical for sustained volatile fatty acids (VFAs) and hydrogen production and significantly reduces solids solubilization. In addition, the development of methanogens in an acidification broth causes failure to the process due to the rapid takeover of methanogens that out compete acidifiers, hydrolyzers and hydrogen producing microorganisms. High rate acidification entails the operation at high concentration of suspended solids (SS) ranging from 10,000 mg/L to 70,000 mg/L i.e. 1% to 7%, and total solids (TS) ranging from 10,000 mg/L to 140000 mg/L i.e. 1% to 14% while operating at short HRTs ranging from 1 hr. to 36 hrs. The use of membrane for biomass retention has been reported in the literature in several studies. However, such studies were either performed using a submerged membrane module in the main process bioreactor, and/or operated in a fed-batch mode, and/or operated on a synthetic feedstock containing primarily soluble carbohydrate i.e. no suspended solids. In addition, there was no control over SRT. The SRT was dictated by the system configuration with respect to bioreactor volume and the efficiency of the membrane in retaining solids.

Previous systems, such as described in WO 2010/085893 describe the use of a gravity settler for decoupling SRT from HRT. In this previous Application, the feedstock is limited to approximately 1% TSS. This limitation of TSS is due to the use of a gravity settler having low suspended solids separation efficiency as the feedstock % TSS is increased beyond 1%. This results in insufficient control of the SRT and inability to effectively decouple SRT from HRT exacerbated by the lack of biological activity and subsequent solubilization of suspended solids within the clarifier. In addition, as described in WO 2010/08593, the gravity settler is considered to be, biologically, an inactive vessel. Thus, the SRT is estimated neglecting any biomass inventory in the gravity settler vessel. In other words, there is no acidification of organic suspended solids in the gravity settler.

Therefore, it would be advantageous to provide a method and system for high rate acidification which decouples the solids residence time (SRT) from the hydraulic retention time (HRT) in order to avoid some of the aforementioned unwanted limitations.

### SUMMARY

In one aspect there is provided a system for high rate acidification, organic solids solubilization, and biohydrogen production, comprising:

a) a high rate acidifier including a completely mixed bioreactor comprising an input for receiving organic stream into said completely mixed bioreactor and an output for discharging an output stream,

wherein the organic stream entering the completely mixed bioreactor is broken down microbiologically by hydrolyzing, acidifying, and hydrogen producing microorganisms to predominantly produce hydrogen gas and carbon dioxide, and a mixture of VFAs and primary alcohols, and wherein hydrogen gas and carbon dioxide are emitted from the completely mixed bioreactor, and wherein the output stream containing the VFAs, primary alcohols and hydrolyzing, acidifying, and hydrogen producing microorganisms is discharged from the completely mixed bioreactor,

b) a membrane unit located downstream of said completely mixed bioreactor comprising one or more microfiltration membranes, and comprising a first side and a second side, the first side comprising a membrane input, a recirculation input, and a membrane concentrate output, the second side comprising a permeate output,

the membrane input on the membrane unit is hydraulically connected with the output of the completely mixed bioreactor for receiving the output stream from said completely mixed bioreactor,

wherein permeate containing predominantly the VFAs and the primary alcohols flow through the one or more microfiltration membranes and is discharged through the membrane permeate output,

wherein the microorganisms in the membrane concentrate output stream are concentrated on the first side of the membrane unit,

c) a storage tank comprising a storage tank input and a storage tank output,

the membrane concentrate output of the membrane unit is hydraulically connected to the storage tank input for receiving concentrated hydrolyzing, acidifying, and hydrogen producing microorganisms from the first side of the membrane unit,

the storage tank output is hydraulically connected to the completely mixed bioreactor for recirculating desired quantities of biomass from the storage tank to said completely mixed bioreactor, and to an output conduit from the storage tank for discharging of excess biomass.

In one example, further comprising a recirculation conduit hydraulically connecting the concentrate output and recirculation input of the membrane unit.

In one example, further comprising a recycling conduit hydraulically connected to the permeate output and the input of the completely mixed bioreactor.

In one example, further comprising temperature controllers associated with the completely mixed bioreactor for controlling a temperature of contents of the completely mixed bioreactor.

In one example, further comprising a dispenser for dispending nutrients and/or pH adjustment compounds into the completely mixed bioreactor.

In one example, wherein the nutrients are any one or combination of nitrogen containing compounds, phosphorous containing compounds, trace metals including iron, manganese, magnesium, calcium, cobalt, zinc, nickel and copper.

In one example, wherein the hydrogen producing microorganisms include any one or combination of C. acetobutyricum, Bacillus thuringiensis, and C. Butyricum.

In one aspect there is provided a method for continuously producing hydrogen gas from a biomass, comprising:

a) seeding a completely mixed bioreactor containing a mixture of microorganisms, the mixture of microorganisms including hydrogen producing microorganisms;

b) continuously flowing an organic stream into the completely mixed bioreactor;

c) using the hydrogen producing microorganisms to continuously break down the biomass in the completely mixed bioreactor and produce hydrogen gas, carbon dioxide gas, and a liquid effluent containing a mixture of volatile fatty acids, primary alcohols, and the mixture of microorganisms;

d) continuously emitting the hydrogen gas and carbon dioxide gas from the completely mixed bioreactor; and

e) decoupling a solid retention time from a hydraulic retention time and controlling the VCF of output stream by flowing the output containing the mixture of volatile fatty acids, the primary alcohols, and the mixture of microorganisms to a microfiltration membrane located downstream of the completely mixed bioreactor, and concentrating the hydrogen producing microorganisms and/or biomass on a first side of said membrane and flowing liquid permeate through said membrane to a second side of the membrane,

f) flowing the concentrated hydrogen producing microorganisms and/or biomass on a first side of said membrane to a storage tank, and recirculating a portion of the microorganisms and/or biomass to the completely mixed bioreactor, and discharging a remaining portion of the biomass from the storage tank in an excess waste stream, and

g) discharging the permeate from the second side of the membrane to a subsequent downstream process and/or partially recycling it to the completely mixed bioreactor.

In one example, including controlling a temperature of completely mixed bioreactor.

In one example, wherein said temperature of the completely mixed bioreactor is maintained in a temperature range from 20° C. to about 70° C.

In one example, comprising dispensing any one or combination of nutrients and pH adjustment compounds into the completely mixed bioreactor.

In one example, wherein the nutrients are any one or combination of nitrogen containing compounds, phosphorous containing compounds, trace metals including iron, manganese, magnesium, calcium, cobalt, zinc, nickel and copper.

In one example, wherein said pH adjustment compounds include, but are not limited to soda ash, sodium bicarbonate, sodium hydroxide, calcium hydroxide, magnesium hydroxide, nitric acid, and hydrochloric acid.

In one example, wherein the hydrogen producing microorganisms include any one or combination of Clostridium acetobutyricum, Bacillus thuringiensis, and Clostridium butyricum.

In one example, wherein the organic stream comprises up to about 15% TSS.

In one example, wherein the SRT is between about 1.6 days to about 4.5 days.

In one example, wherein the HRT is between about 6 hours to about 18 hours.

In one example, wherein the VCF is between about 1.5 to about 2.2.

A further understanding of the functional and advantageous aspects of the invention can be recognized by reference to the following detailed description and drawings.

### BRIEF DESCRIPTION OF THE DRAWING

Embodiments of the present disclosure will now be described, by way of example only, with reference to the attached Figures.

FIG. 1 is a block diagram showing an example of the present system for employing an example of the method described herein.

FIG. 2 is a block diagram showing an example of the present system for employing an example of the method described herein.

FIG. 3 depicts an example of a membrane unit.

### DETAILED DESCRIPTION

As noted above, previous systems, such as described in WO 2010/085893 describe the use of a gravity settler for decoupling SRT from HRT. In this previous Application, the feedstock is limited to approximately 1% TSS. This limitation of TSS is due to the use of a gravity settler having low suspended solids separation efficiency as the feedstock % TSS is increased beyond 1%. This results in insufficient control of the SRT and inability to effectively decouple SRT from HRT.

There is described herein a system and method to achieve improved control of the SRT for feedstock containing up to 15% TSS to achieve superior solubilization of suspended solids ranging from 15% to 65% of the % TSS in the feedstock as well as significant productivity in Volatile Fatty Acids (VFAs) ranging from 0.1 mg VFAs/mg TSS to 0.4 mg VFAs/mg TSS_{feed} increase over the VFAs contained in the feedstock. Internal recirculation of permeate stream which has very low suspended solids allows the processing of high suspended solids feedstocks of up to 15% TSS.

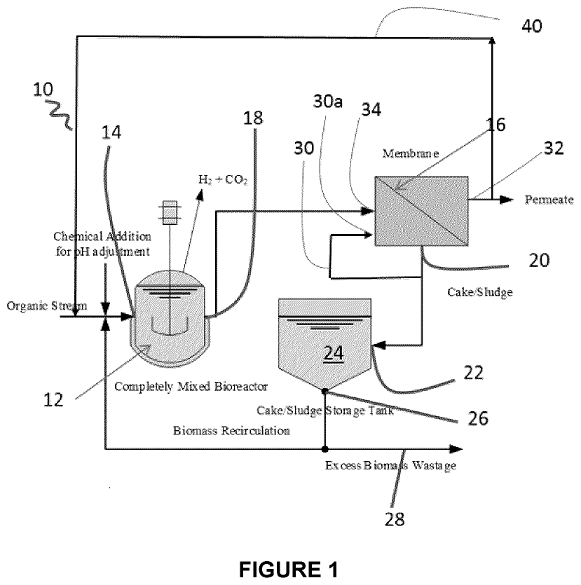

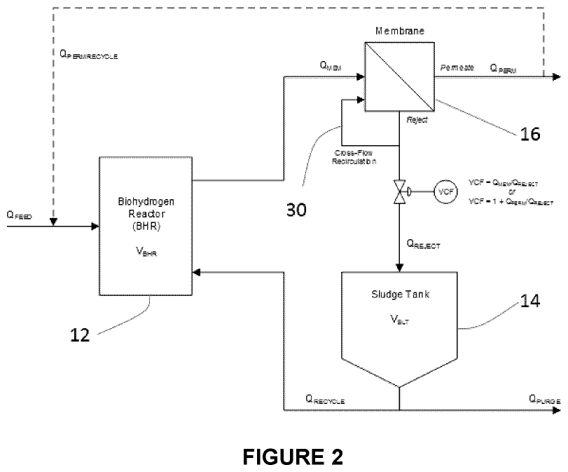

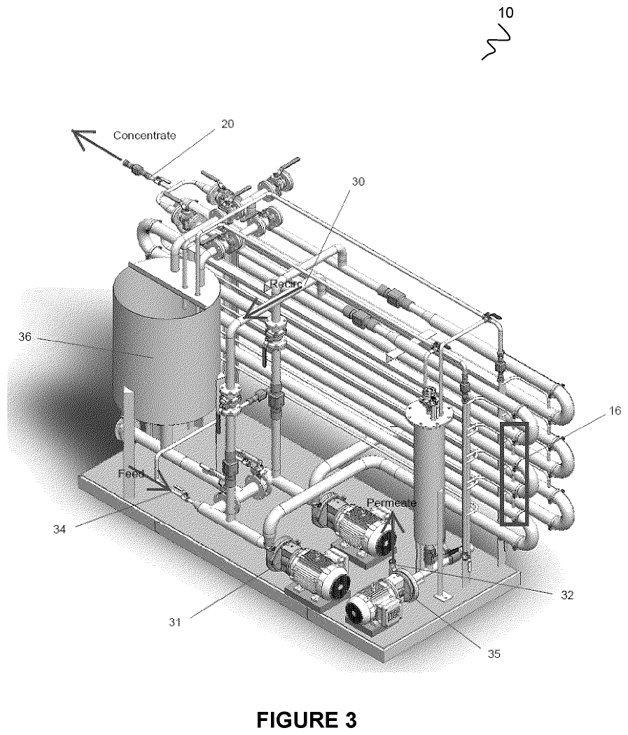

FIGS. 1 and 2 depicts an example of system (10) for high rate acidification, organic solids solubilization, and biohydrogen production from organic streams (also referred to as feedstock). FIG. 3 depicts an example of a membrane unit.

System (10) comprises completely mixed bioreactor (12), a membrane unit (16) and cake/sludge storage/acidification tank (24).

It has been determined that using both a membrane unit (16) and a cake/sludge storage/acidification tank (24) in combination, resulted in a surprisingly high solubilization rate in addition to hydrogen production compared to the gravity settler technology that did not offer any noticeable solubilization. The cake/sludge storage/acidification tank is required for process control as well as the unexpected and positive advantage of increased solubilization.

As used herein, the phrase “completely mixed bioreactor” refers to a mechanically or hydraulically agitated vessel including microorganisms in suspension and a growth media, typically comprised of nutrients such as organic carbon, nitrogen-containing compounds, phosphorous-containing compounds, and trace mineral solutions.

The cake/sludge storage/acidification tank is, biologically, an active vessel. The tank contains a high population of hydrolyzing, acidifying, and hydrogen producing microorganisms and is designed to operate in a plug-flow mode or continuously stirred tank reactor mode.

As used herein, the phrase “organic stream” refers to streams that include carbon and hydrogen such as, but are not limited to, alcohols, ketones aldehydes, volatile fatty acids, esters, carboxylic acids, ethers, carbohydrates, proteins, lipids, polysaccharides, monosaccharide, cellulose, and nucleic acids.

Organic streams may be obtained from one or more feed stocks including, but is not limited to, thin stillage from a corn-based ethanol plant, municipal source separated organics, municipal sewage sludge, and various industrial organic wastes.

Referring to the specific examples of the Figures, system (10) comprises a high rate acidifier including a completely mixed bioreactor (12) having an input (14) and an output (18). Input (14) is for receiving an organic stream into said completely mixed bioreactor (12). Output (18) is for discharge of an output of a reaction product from the completely mixed bioreactor (12).

Membrane unit (16) comprises one or more microfiltration membranes, is located downstream of said completely mixed bioreactor (12), and comprises a first side and a second side. The first side comprises membrane input (34), recirculation input (30a), and membrane concentrate output (20). The second side comprises permeate output (32).

Membrane unit input (34) is for receiving an output from said completely mixed bioreactor (14). Membrane unit (16) is hydraulically connected to mixed bioreactor (12) via output (18) of said completely mixed bioreactor (14) and membrane unit input (34) of membrane unit (16).

Optionally, permeate output is hydraulically connected to input (14) via recycling conduit (40).

Cake/sludge storage/acidification tank (24) comprises cake/sludge storage/acidification tank input (22) and cake/sludge storage/acidification tank output (26).

Membrane concentrate output (20) is hydraulically connected to cake/sludge storage/acidification tank input (22) of cake/sludge storage/acidification tank (24).

Optionally, recirculation conduit (30) recirculates fluid from membrane concentrate output (20) to recirculation input (30a) on the first side of said membrane unit (16). This recirculation may reduce fouling. Fouling can be reversed through a scheduled clean in place (CIP) (36) for short periods of time.

Cake/sludge storage/acidification tank output (26) is hydraulically connected to completely mixed bioreactor (14) for recirculating desired quantities of biomass from cake/sludge storage/acidification tank (24) to said completely mixed bioreactor (14), and including an output conduit (28) from the bottom of said cake/sludge storage/acidification tank (24), for discharging of excess biomass.

Membrane unit (16) comprises one or more microfiltration membranes, and permits separation using microfiltration (also referred to as MF), wherein a fluid is passed through the microfiltration membrane to separate microorganisms and suspended particles from a process liquid. The microfiltration membrane comprises a plurality of pores.

The pore size of the microfiltration membrane selected may vary with conditions.

In some examples, the pore size of the microfiltration membrane is selected to prevent 99% or more of the suspended solids and bacteria in the microfiltration membrane feed from passing from the first side of membrane unit (16) through membrane unit (16), and large enough to allow flow of permeate through the membrane with minimal pressure drop across the membrane.

Particles such as water, monovalent ions (e.g. sodium, chloride), dissolved organic matter and small colloids pass through the pores of the membrane.

The selection of the preferred pore size of the membrane is dependent upon the particle size distribution of the feed to the membrane, the size of the bacteria, and/or the ease of which liquid is removed from the membrane feed, and the like.

In some examples, the pore size of the membrane is in range of from 0.1 μm to 10 μm, and separates suspended particles and large bacteria from the process fluid. In some examples, the pore size of the membrane is in the range of about 0.1 μm to 0.4 μm.

Membranes may be made from a variety of materials, including, but not limited to organic membranes and/or inorganic membranes.

Organic membranes may be made from materials such as, but not limited to, cellulose acetate (CA), polysulfone (PS), polyvinylidene fluoride (PVDF), polyethersulfone (PES) and polyimide (PI).

Inorganic membranes may be made from materials such as, but not limited to, ceramic and/or various sintered metals.

Microfiltration membranes may be fabricated into spiral wound units or tubular units. Typically, tubular membrane units are used when handling liquids with higher amounts of suspended solids material.

Continuous operation microfiltration membranes typically operate in a cross-flow filtration mode, where the process fluid is recirculated across the membrane surface in order to reduce fouling. Fouling can be reversed through a scheduled clean in place (CIP) for short periods of time.

FIG. 2 depicts an example of a process configuration for the system described herein, and comprises completely mixed bioreactor (12), membrane unit (16), and cake/sludge storage/acidification tank (14).

FIG. 3 depicts an example of a membrane unit, comprised of a typical redundant 4 membrane module arrangement (16). Membrane crossflow recirculation (30) is accomplished with a recirculation pump (31) and permeate product (32) is discharged from the system using a pump (35). The concentrated output stream (20) is discharged from the system using the pressure from the recirculation pump (31). A control device on the concentrate outlet stream (20) maintains a backpressure on the recirculation pump (31) to provide sufficient driving force to extract permeate through the membrane modules (16). The feed stream (34) is introduced into the recirculation stream (30) at a controlled rate. The recirculation pump (31) flow is controlled to reduce membrane module (16) fouling. Periodically the membranes require cleaning and a CIP system (36) is utilized.

The volumetric concentration factor (VCF) is a controlled variable, and its maximum attainable value is a function of the following: the maximum % TSS in the reject stream which is transferable to downstream unit operations respecting the limitations of the material handling equipment, for example, with a centrifugal pump this could be in the range of 18-20% TSS; the % TSS solubilization occurring in the process; the % TSS in the incoming feed stream.

Permeate recycle is only required when the incoming feed stream is high in % TSS (typically >8% TSS) and the system cannot maintain the required VCF to achieve the desired SRT.

The main controlled parameters for the process are the SRT and HRT, with their associated equations shown below:

$$SRT = \frac{{V}_{BHR} * {TSS}_{BHR} + {V}_{SLT} * {TSS}_{SLT}}{{Q}_{PERM} * {TSS}_{PERM} + {Q}_{PURGE} * {TSS}_{SLT}} \qquad ( equation 1 )$$

The permeate stream, due to the nature and pore-size of the membrane will have a negligible amount of suspended solids, and as such:

$$T S {S}_{P E R M} \approx 0 and VCF = \frac{{TSS}_{SLT}}{{TSS}_{BHR}}$$

The SRT equation, simplifies to:

$$H R T = \frac{{V}_{B H R}}{{Q}_{FEED}} * 24 \qquad ( equation 3 )$$

Where;

SRT=solids retention time, days, described as the mass of suspended solids retained in the system divided by the rate of suspended solids mass leaving the system.

HRT=hydraulic retention time, hours.

VBHR=biohydrogen reactor controlled volume, m³.

VSLT=cake/sludge storage/acidification tank controlled volume, m³.

QFEED=flow of feedstock to the BHR, m³/d.

QRECYCLE=flow of sludge from cake/sludge storage/acidification tank to BHR, m³/d.

VCF=volumetric concentration factor

The method to effectively control the SRT and HRT is described by equations 2 and 3, above.

For a given flow of feedstock (Q_{FEED}), the controlled volume of the biohydrogen reactor is determined by the required HRT. To establish the desired SRT, a combination of sludge recycle (Q_{RECYCLE}), VCF and controlled sludge volume (VSLT) is required. The membrane capacity (i.e. surface area) needs to be chosen to handle the sum of the feedstock (Q_{FEED}) and sludge recycle (Q_{RECYCLE}).

As apparent from the SRT equation, the cake/sludge storage/acidification tank is essential for achieving the optimum SRT in the system, and maintaining process stability during any organic or hydraulic shock loads. Without a cake/sludge storage/acidification tank, there is no control of the SRT.

The membrane VCF is controlled to achieve the desired solids concentration in the membrane reject stream, providing an accurate SRT control to deliver the significantly improved performance in suspended solids solubilization efficiency and volatile fatty acids productivity compared to gravity settling using clarifiers.

In contrast, in those applications using a clarifier, the only means to control SRT is through controlling the sludge wastage flow rate. Also, in a clarifier, the concentration of suspended solids in the supernatant, recycle stream or purge stream is dictated by the settling efficiency of solids which is dependent of the physical properties of the solids. As the clarifier is considered an inactive vessel, the volume of sludge at the bottom of the clarifier, if any, is neglected when estimating the SRT.

In order to achieve the desired performance for conversion of suspended solids into VFAs, the SRT can be controlled in a range of 1.6 to 4.5 days, preferably in the range of 1.8 to 2.5 days. The HRT can be controlled in a range of 6 to 18 hours, preferably in the range of 8 to 16 hours.

The VCF operating set point will be dependent upon the nature and physical characteristics of the feedstock, but will typically be in the range of 1.5 to 2.2.

As an example, increasing the sludge tank storage volume will allow operation at a lower suspended solids concentration in the sludge tank to maintain the desired SRT which translates to a lower VCF setpoint for the same % TSS in the membrane feed.

In one example, to maximize the performance of the acidification process, the system is operated at an HRT between 1 hr to 36 hrs. The SRT is controlled between 1.6 days to 4.5 days. A storage tank (24) is located downstream of the membrane unit (16). The storage tank (24) volume is designed to offer the desired SRT in the system through offering an inventory of highly active bacterial consortium, which may include but are not limited to, C. acetobutyricum, Bacillus thuringiensis, and/or C. butyricum, the sludge storage tank is essential for achieving the optimum SRT in the system, and maintaining process stability during any organic or hydraulic shock loads. Without a storage tank, there is no control of the SRT. The membrane VCF is controlled to achieve the desired solids concentration in the concentrate. The concentrate stream is fed to the sludge storage tank. The SRT is accurately controlled using a sludge wastage pump and sludge recycle pump connected to the bottom of the sludge storage tank (24). In some examples, the pump may include centrifugal, progressive cavity, piston or gear pumps.

The system operates on feedstocks of high SS concentrations up to about 70,000 mg/L, i.e. about 7% wt/vol, and TS up to about 144,000 mg/L, ie about 14% wt/vol %. In one example, the feedstocks SS concentrations are in the range of about 10,000 mg/L to 70,000 mg/L i.e. 1% wt/vol % to 7% wt/vol %, and TS ranging from 10,000 mg/L to 140000 mg/L i.e. 1% to 14% while operating at short HRTs ranging from 1 hr to 36 hrs. The sludge storage tank contains an inventory of concentrated acidifying bacterial consortium that is retained using the membrane unit. The concentration of SS in the sludge storage tank (24) is controlled and can be as high as 18%. The TS concentration in the sludge storage tank (24) can reach 20%. The control of SRT between 1.6 days to 4.5 days is achieved by: the control of the membrane VCF, the flow rate of excess biomass wastage pump, and the flow rate of biomass recycle pump. Depending on the feedstock SS concentration and particle size, COD levels and composition, the HRT and SRT of the system are adjusted according to the process HRT and SRT ranges defined above.

In one example, in operation, the organic stream (labelled organic stream in FIG. 1) entering the completely mixed bioreactor (12) is broken down microbiologically by hydrolyzing, acidifying, and hydrogen producing microorganisms, which may include but are not limited to, C. acetobutyricum, Bacillus thuringiensis, and/or C. butyricum to predominantly hydrogen gas and carbon dioxide, and a mixture of volatile fatty acids and primary alcohols in the completely mixed bioreactor (12). The hydrogen gas (H₂) and carbon dioxide (CO₂) are emitted from the completely mixed bioreactor (12), and a liquid effluent containing the volatile fatty acids, primary alcohols and hydrolyzing, acidifying, and hydrogen producing microorganisms flow from output (18) of completely mixed bioreactor 12 to membrane unit (16).

As used herein, the phrase “hydrolyzing, acidifying, and hydrogen producing microorganisms” means microorganisms capable of fermenting organics under anaerobic conditions to produce hydrogen, carbon dioxide, and a variety of organic acids and alcohols.

When in membrane unit (16) the microorganisms are concentrated on the first side of the membrane unit (16) and sent to storage tank (24) through membrane concentrate output (20).

Liquid permeate containing predominantly the volatile fatty acids and the primary alcohols flow from second side of the membrane unit (16) through permeate output (32) into any subsequent process (not shown) or may be partially recycled back to the front-end feed stream via recycling conduit (40), and wherein concentrated hydrolyzing, acidifying, and hydrogen producing microorganisms and biomass/suspended solids are sent to storage tank (24), from which, they are recirculated back to the completely mixed bioreactor (12).

The system also preferably includes temperature controllers for controlling the temperature in the completely mixed bioreactor (12). A typical temperature range in which the temperature of the contents of bioreactor (12) is maintained between from about 20° C. to about 70° C.

The system may also include a dispenser (not shown) for dispensing nutrients and pH adjustment compounds into the completely mixed bioreactor (14). The nutrients may be, but are not limited to, any one or combination of nitrogen containing compounds, phosphorous containing compounds, trace metals including iron, manganese, magnesium, calcium, cobalt, zinc, nickel, and copper. The pH adjustment compounds include, but are not limited to soda ash, sodium bicarbonate, sodium hydroxide, calcium hydroxide, magnesium hydroxide, nitric acid, and hydrochloric acid.

Examples of hydrolyzing, acidifying, and hydrogen generating microorganisms include, but are not limited to, C. acetobutyricum, Bacillus thuringiensis, and C. butyricum.

Examples of pH adjustment compounds include, but are not limited to soda ash, sodium bicarbonate, sodium hydroxide, calcium hydroxide, magnesium hydroxide, nitric acid, and hydrochloric acid.

To gain a better understanding of the invention described herein, the following examples are set forth. It should be understood that these examples are for illustrative purposes only. Therefore, they are not intended to nor should they limit the scope of this invention in any way.

### Examples

A demonstration scale version of the system has been operated in continuous mode 24 hours/day/7 days/week for 8 months using thin stillage from a corn-based ethanol plant. The system capacity is 1 tonne of dry solids per day. The system consists of a completely mixed bioreactor of 5.5 m³ volume, a sludge tank of 5 m³ volume, and four cross-flow membrane modules with 0.2 microns pore size. The membrane performs separation at a microfiltration level. The membrane has sufficient flexibility whereby the concentration of the concentrate can be controlled via a parameter on the membrane skid called the volumetric concentration factor (VCF).

Regulation of the membrane VCF has an impact on SRT. There is a significant hold up volume of concentrated sludge in the sludge tank with use of the membranes. A higher VCF increases the suspended solids concentration in the concentrate, which in turn would increase SRT. Additionally, if the concentrate purge from the storage tank is regulated or minimized the SRT can be further increased or regulated as required. During both mesophilic and thermophilic operation, the system has been operated at HRT of 12 hrs. The SRT has been controlled at 3 days. The SRT was controlled using a sludge wastage pump and sludge recycle pump connected to the bottom of the sludge storage tank, and by adjusting the membrane VCF between 1.8 to 2.4. All of the permeate from the membrane was discharged to a downstream process with none being recycled to the front-end feed stream. The system is equipped with heat exchangers, and has been operated at mesophilic temperature of 37° C. for 12 weeks, then thermophilic temperature of 55° C. for 12 weeks. The pH in the bioreactor has been maintained between 5.2-5.6.

The system was monitored daily and samples from various process streams were collected for analysis 3 times per week. The samples were analyzed for Total chemical oxygen demand (TCOD), Soluble chemical oxygen demand (SCOD), Total Solids (TS), Volatile solids (VS), Total suspended solids (TSS), Volatile suspended solids (VSS), and Total volatile fatty acids (TVFAs). Biogas mass was measured continuously using a mass flow meter. Biogas composition was analyzed using a gas chromatograph (SRI 8610C, SRI instruments, Torrance, Calif.) with a thermal conductivity detector (TCD) temperature of 60° C. and a molecular sieve column (Mol Sieve 6; mesh 80/100, 6 ft., ⅛ in.) at a temperature of 150° C. Nitrogen (99.999%, PraxAir, Canada) was used as carrier gas at a flow rate of 20 mL/min.

TVFAs, TCOD and SCOD were measured using UV-Vis spectrophotometer (DR6000, HACH, Canada). The various components of VFAs were analyzed using a GC equipped with a flame ionization detector (FID) (SRI 8610C, SRI instruments, Torrance, Calif.) and a MXT-WAX capillary column (30 m×0.53 mm, ID 0.53 mm, Restek Co., USA). The initial temperature of the column oven was 80° C., and it was increased to 180° C. with a temperature gradient of 3° C./min. The temperature of the detector was set at 200° C. Helium (99.999%, PraxAir, Canada) was used as a carrier gas at a constant pressure of 8 psi. Hydrogen and air (PraxAir, Canada) were used for a FID at constant flows of 25 and 250 mL/min, respectively. The GC-FID was calibrated using a standard solution (46975-U, Sigma-Aldrich, Canada). All samples were acidified with phosphoric acid (PX0996-6, HPLC grade, EM Science, USA) and filtrated using a syringe filter (Hydrophilic PTFE Syringe Filters, 0.2 μm, Acrodisc, USA) prior to analysis. In addition, TSS and VSS concentrations were measured using standard methods [APHA, 1995], while soluble parameters were analyzed after filtering the samples through 0.45 μm filter paper. Solids and TCOD mass balances were performed on a weekly basis for calculations of solubilization efficiency, VFAs productivity, solids retention time, and other necessary process key performance indicators (KPIs).

The system has been started up using anaerobic sludge from a secondary digester located at a municipal wastewater treatment plant in Ontario. At start-up, the sludge was preheated to 70° C. to inhibit methanogenic activity. Thin stillage from the corn-based ethanol plant was characterized by the following; TCOD of 110-150 kg/m3, SCOD of 50 kg/m3-70 kg/m3, TS of 60-80 kg/m3, VS of 55-75 kg/m3, TSS of 35-50 kg/m3, VSS of 32-45 kg/m3, TVFAs of 0.5-3 kg/m3, and pH of 3-4.

### Mesophilic Operation

After 8 days from start-up, the system reached steady-state conditions and was operated for 8 weeks at steady-state conditions. The SS solubilization efficiency ranged from 32% to 46% with an average of 40%. The concentration of total volatile fatty acids (TVFAs) in the permeate ranged from 10,000 mg/L to 15,000 mg/L with an average of 12,500 mg/L. Acetate was the primary constituent of VFAs reaching concentrations of up to 5,000 mg/L. The average biogas production was 50 kg/day. Hydrogen concentration in the biogas ranged between 60% to 70% by volume and the balance was carbon dioxide. There was no detection of any methane gas throughout the mesophilic operation.

### Thermophilic Operation

The system temperature was gradually increased from 37° C. to 55° C. over a period of 2 weeks. The system reached steady-state conditions after one week of operation at 55° C. and was operated for 8 weeks at steady-state conditions. The SS solubilization efficiency ranged from 30% to 45% with an average of 38%. The concentration of total volatile fatty acids (TVFAs) in the permeate ranged from 9,000 mg/L to 14,000 mg/L with an average of 11,500 mg/L. Acetate was the primary constituent of VFAs reaching concentrations of up to 4,500 mg/L. The average biogas production was 60 kg/day. Hydrogen concentration in the biogas ranged between 65% to 70% by volume and the balance was carbon dioxide. There was no detection of any methane gas throughout the mesophilic operation.

Specific Biohydrogen Production Rate Tests for the Cultures Collected from the Demonstration System.

A total of 63 samples in 21 sets from the demo system were collected from various locations in the process. The samples were all characterized for TSS, VSS, total and soluble carbohydrates. Biohydrogen potential tests were conducted on the various sets of samples to assess the system selectively and ability to enrich biohydrogen producing and acidifying bacteria, as reflected by the maximum biomass-specific hydrogen production rates (MSHPR). The membrane demonstrated excellent selectivity for biohydrogen producing bacteria with the MSHPR of 86.3±42.1 mL H2/gVSS·h more than three times higher than the reactor (25.6±11.4 mL H2/gVSS·h), and the permeate (29.1±13.5 mL H2/gVSS·h). There was no detection of any methanogenic activity throughout the testing evident from the absence of any methane gas production.

The steady operation of the system on thin stillage for over 20 weeks of continuous steady-state operation at both mesophilic and thermophilic conditions is attributed to the use of the membrane and cake/sludge storage tank. The results have indicated that more than 40% solubilization in suspended solids could be achieved and concentrations of TVFAs as high as 15,000 mg/L can be attained. The bacterial culture activity test confirmed the unique capabilities of the system in concentrating and enriching hydrolyzing, acidifying, and hydrogen producing microorganisms. The high concentration of hydrolyzing, acidifying, and hydrogen producing microorganisms in the sludge storage tank during both mesophilic and thermophilic operation resulted in about 80% to 90% of the solubilization of suspended solids occurring in the cake/sludge storage/acidification tank while only 10% to 20% of the solubilization of suspended solids is occurring in the biohydrogen reactor.

An example of solubilization data on the cake/sludge storage/acidification tank and comparative values against the BHR is shown for Mesophilic operation (37° C.) (Table 1) and Thermophilic Operation (53° C.) (Table 2). It is evident from the steady-state data of the system during both mesophilic and thermophilic operation that about 80% to 90% of the solubilization of suspended solids is occurring in the cake/sludge storage/acidification tank while only 10% to 20% of the solubilization of suspended solids is occurring in the biohydrogen reactor.

An estimate of the BHR solubilization efficiency is based on the analytical total suspended solids (TSS) measurements and flows in the first stage. Grab Samples representing an analytical snapshot of the first stage operating system are taken. The TSS of the Organic Stream from the offloading (day) tank, the Biomass Recirculation from bottom of the Cake/Sludge Storage Tank and the effluent from the Completely Mixed Bioreactor (BHR) form part of the Grab Samples collected and are analyzed in an offsite laboratory. These analyses coupled with the flows in the first stage are used to estimate the TSS concentration for a theoretical mixed stream termed the TSS_{BHR FEED} This is a mixture of the First Stage Feed and the Sludge Storage Tank Recycle Stream entering the BHR as shown in equation 2. Once this is estimated, the Solubilization BHR (%) is then calculated as shown in Equation 1.

$$\begin{matrix}Solubilization BHR ( \% ) = \frac{T S {S}_{BHR FEED} - T S {S}_{BHR EFFLUENT}}{T S {S}_{BHR FEED}} & ( Equation 4 ) \\ TS {S}_{BHR FEED} = \frac{{Q}_{F E E D} * T S {S}_{F E E D} + {Q}_{R E C Y C L E} * T S {S}_{R E C Y C L E}}{{Q}_{F E E D} + {Q}_{R E C Y C L E}} & ( Equation 5 )\end{matrix}$$

### For Example:

For the average data presented in table 1 below:

### Given:

Q_{FEED}=11.9 m3/day
Q_{RECYCLE}=3.9 m3/day
TSS_{FEED}=43310 mg/I
TSS_{BHR EFFLUENT}=56540 mg/I
TSS_{RECYCLE}=118640 mg/I

### Solution:

### Using Equation 4 & 5,

$$T S {S}_{BHR FEED} = \frac{11.907 * 43310 + 3.872 * 118640}{11.907 + 3.872} \times 100 \% = 61582 mg / l$$

$$Solubilization BHR ( \% ) = \frac{61818 - 56540}{61582} \times 100 \% = 9 \%$$

The solubilization of the first stage is calculated as the difference of the total suspended solids entering the system versus that leaving the first stage of the system. Meanwhile, the solubilization of the storage tank is the difference between the First Stage solubilization and the BHR solubilization:

$$\begin{matrix}Solubilization First Stage ( \% ) = \frac{{Q}_{FEED} {TSS}_{FEED} - {Q}_{PERM} {TSS}_{PERM} - {Q}_{PURGE} {TSS}_{PURGE}}{{Q}_{FEED} {TSS}_{FEED}} \times 100 \% & ( Equation 6 ) \\ Solubilization Storage Tank ( \% ) = Solubilization First Stage ( \% ) - Solubilization BHR ( \% ) & ( Equation 7 )\end{matrix}$$

### Given:

Q_{FEED}=11.9 m3/day
Q_{RECYCLE}=3.9 m3/day
Q_{PERMEATE}=8.87 m3/day
Q_{PURGE}=2.69 m3/day
TSS_{FEED}=43310 mg/I
TSS_{BHR EFFLUENT}=56540 mg/I
TSS_{RECYCLE}=TSS_{PURGE}=118640 mg/I
TSS_{PERMEATE}=266 mg/I

### Solution:

$$Using Equation 6 , \\ Solubilization First Stage ( \% ) = \frac{11.9 * 43310 - 8.87 * 266 - 2.69 * 118640}{11.9 * 43310} \times 100 \% = 38 \%$$

### Using Equation 7,

Solubilization Storage Tank (%)=38%−9%=29%

Therefore, the ratio of solubilization between the BHR and the Storage tank can be calculated as follows

BHR Solubilization ratio=9/(29+9)=23%

Sludge Storage Tank Solubilization ratio=29/(29+9)=77%

TABLE 1
Mesophilic Operation (37° C.)
Mesophilic Operation (37° C.)

| | | Daily | Daily | | | | | | | |
|---|---|---|---|---|---|---|---|---|---|---|
| | | Sludge | Sludge | | TSS | | | | | |
| | | Storage | Storage | | Sludge | | | | | Sludge |
| | Daily BHR | Tank | Tank | | Storage | | | | Overall | storage tank |
| | Totalized | Recycle | Recycle | TSS | Tank | TSS BHR | | Solubilization | solubilization | solubilization |
| | Feed Flow | Flow | Flow | Offloading | recycle | effluent | TSS BHR | efficiency | efficiency | efficiency |
| | (m3/day) | (kg/day) | (m3/day) | tank (mg/l) | (mg/l) | (mg/l) | Feed (mg/l) | of BHR (%) | (%) | (%) |
| Average | 11.9 | 3920 | 3.9 | 43310 | 118640 | 56540 | 61818 | 9% | 38% | |

| | | |
|---|---|---|
| Ratio of Solubilization | BHR | Sludge Storage Tank |
| | 23% | 77% |

TABLE 2
Thermophilic Operation (53° C.)
Thermophilic Operation (53° C.)

| | | Daily | Daily | | | | | | | |
|---|---|---|---|---|---|---|---|---|---|---|
| | | Sludge | Sludge | | TSS | | | | | |
| | | Storage | Storage | | Sludge | | | | | sludge |
| | Daily BHR | Tank | Tank | | Storage | | | | | storage tank |
| | Totalized | Recycle | Recycle | TSS | Tank | TSS BHR | | Solubilization | Overall | solubilization |
| | Feed Flow | Flow | Flow | Offloading | recycle | effluent | TSS BHR | efficiency | solubilization | efficiency |
| | (m3/day) | (kg/day) | (m3/day) | tank (mg/l) | (mg/l) | (mg/l) | Feed (mg/l) | of BHR (%) | efficiency(%) | (%) |
| Average | 10 | 3212 | 3.2 | 48877 | 114497 | 62400 | 64889 | 4% | 39% | |

| | | |
|---|---|---|
| Ratio of Solubilization | BHR | Sludge Storage Tank |
| | 10% | 90% |

### REFERENCES

Chang, J. S., Lee, K. S., and Lin, P. J., (2002) Biohydrogen production with fixed-bed bioreactors. Int. J. Hydrogen Energy 27 (11/12), 1167-1174.
Das, D., Khanna, N., Veziroglu, T. N., (2008) Recent developments in biological hydrogen production processes. Chem Ind. And chem. Eng. 14 (2), 57-67.
Horiuchi J. I., Shimizu T., Tada K., Kanno T., Kobayashi M., (2002) Selective production of organic acids in anaerobic acid reactor by pH control. Bioresource Technol 82, 209-13.
Li, C., Fang, H. H. P., (2007) Fermentative hydrogen production from wastewater and solid wastes by mixed cultures. Critical reviews in Env. Sci. and Tech., 37, 1-39.
Matsunaga, T., Hatano, T., Yamada, A., Matsumoto, M., (2000) Microaerobic hydrogen production by photosythetic bacteria in a double phase photobioreactor. Biotechnol. Bioeng. 68 (6), 647-651.
Wu, S. Y., Hung, C. H., Lin, C. Y., Lin, P. J., Lee, K. S., Lin, C. N., Chang, F. Y. And Chang, J. S. (2008) HRT-dependent hydrogen production and bacterial community structure of mixed anaerobic microflora in suspended, granular and immobilized sludge systems using glucose as the carbon substrate. Int. J. Hydrogen Energy 33, 1542-1549.
Zhang, H., Mary, A. B., Bruce, E. L., (2006) Biological hydrogen production by Clostridium acetobutylicum in an unsaturated flow reactor. Water Research 40, 728-734.

The embodiments described herein are intended to be examples only. Alterations, modifications and variations can be effected to the particular embodiments by those with skill in the art. The scope of the claims should not be limited by the particular embodiments set forth herein, but should be construed in a manner consistent with the specification as a whole.

All publications, patents and patent applications mentioned in this Specification are indicative of the level of skill those skilled in the art to which this invention pertains and are herein incorporated by reference to the same extent as if each individual publication patent, or patent application was specifically and individually indicated to be incorporated by reference.

The invention being thus described, it will be obvious that the same may be varied in many ways. Such variations are not to be regarded as a departure from the spirit and scope of the invention, and all such modifications as would be obvious to one skilled in the art are intended to be included and are included within the scope of the following claims.

What is claimed is:

1. A system for high rate acidification, organic solids solubilization, and biohydrogen production, comprising:
a) a high rate acidifier including a completely mixed bioreactor comprising an input for receiving organic stream into said completely mixed bioreactor and an output for discharging an output stream,
wherein the organic stream entering the completely mixed bioreactor is broken down microbiologically by hydrolyzing, acidifying, and hydrogen producing microorganisms to predominantly produce hydrogen gas and carbon dioxide, and a mixture of VFAs and primary alcohols, and wherein hydrogen gas and carbon dioxide are emitted from the completely mixed bioreactor, and wherein the output stream containing the VFAs, primary alcohols and hydrolyzing, acidifying, and hydrogen producing microorganisms is discharged from the completely mixed bioreactor,
b) a membrane unit located downstream of said completely mixed bioreactor comprising one or more microfiltration membranes, and comprising a first side and a second side, the first side comprising a membrane input, a recirculation input, and a membrane concentrate output, the second side comprising a permeate output,
the membrane input on the membrane unit is hydraulically connected with the output of the completely mixed bioreactor for receiving the output stream from said completely mixed bioreactor,
wherein permeate containing predominantly the VFAs and the primary alcohols flow through the one or more microfiltration membranes and is discharged through the membrane permeate output,
wherein the microorganisms in the membrane concentrate output stream are concentrated on the first side of the membrane unit,
c) a storage tank comprising a storage tank input and a storage tank output,
the membrane concentrate output of the membrane unit is hydraulically connected to the storage tank input for receiving concentrated hydrolyzing, acidifying, and hydrogen producing microorganisms from the first side of the membrane unit,
the storage tank output is hydraulically connected to the completely mixed bioreactor for recirculating desired quantities of biomass from the storage tank to said completely mixed bioreactor, and to an output conduit from the storage tank for discharging of excess biomass.

2. The system of claim 1, further comprising a recirculation conduit hydraulically connecting the concentrate output and recirculation input of the membrane unit.

3. The system of claim 1 or 2, further comprising a recycling conduit hydraulically connected to the permeate output and the input of the completely mixed bioreactor.

4. The system according to any one of claims 1 to 3, further comprising temperature controllers associated with the completely mixed bioreactor for controlling a temperature of contents of the completely mixed bioreactor.

5. The system according to any one of claims 1 to 4, further comprising a dispenser for dispending nutrients and/or pH adjustment compounds into the completely mixed bioreactor.

6. The system according to claim 5, wherein the nutrients are any one or combination of nitrogen containing compounds, phosphorous containing compounds, trace metals including iron, manganese, magnesium, calcium, cobalt, zinc, nickel and copper.

7. The system according to any one of claims 1 to 6, wherein the hydrogen producing microorganisms include any one or combination of C. acetobutyricum, Bacillus thuringiensis, and C. Butyricum.

8. A method for continuously producing hydrogen gas from a biomass, comprising:
a) seeding a completely mixed bioreactor containing a mixture of microorganisms, the mixture of microorganisms including hydrogen producing microorganisms;
b) continuously flowing an organic stream into the completely mixed bioreactor;
c) using the hydrogen producing microorganisms to continuously break down the biomass in the completely mixed bioreactor and produce hydrogen gas, carbon dioxide gas, and a liquid effluent containing a mixture of volatile fatty acids, primary alcohols, and the mixture of microorganisms;
d) continuously emitting the hydrogen gas and carbon dioxide gas from the completely mixed bioreactor; and
e) decoupling a solid retention time from a hydraulic retention time and controlling the VCF of output stream by flowing the output containing the mixture of volatile fatty acids, the primary alcohols, and the mixture of microorganisms to a microfiltration membrane located downstream of the completely mixed bioreactor, and concentrating the hydrogen producing microorganisms and/or biomass on a first side of said membrane and flowing liquid permeate through said membrane to a second side of the membrane,
f) flowing the concentrated hydrogen producing microorganisms and/or biomass on a first side of said membrane to a storage tank, and recirculating a portion of the microorganisms and/or biomass to the completely mixed bioreactor, and discharging a remaining portion of the biomass from the storage tank in an excess waste stream, and
g) discharging the permeate from the second side of the membrane to a subsequent downstream process and/or partially recycling it to the completely mixed bioreactor.

9. The method according to claim 8 including controlling a temperature of completely mixed bioreactor.

10. The method according to claim 8 or 9, wherein said temperature of the completely mixed bioreactor is maintained in a temperature range from 20° C. to about 70° C.

11. The method according to any one of claims 8 to 10, comprising dispensing any one or combination of nutrients and pH adjustment compounds into the completely mixed bioreactor.

12. The method according to claim 11, wherein the nutrients are any one or combination of nitrogen containing compounds, phosphorous containing compounds, trace metals including iron, manganese, magnesium, calcium, cobalt, zinc, nickel and copper.

13. The method according to claim 11, wherein said pH adjustment compounds include, but are not limited to soda ash, sodium bicarbonate, sodium hydroxide, calcium hydroxide, magnesium hydroxide, nitric acid, and hydrochloric acid.

14. The method according to any one of claims 8 to 13, wherein the hydrogen producing microorganisms include any one or combination of Clostridium acetobutyricum, Bacillus thuringiensis, and Clostridium butyricum.

15. The method according to any one of claims 8 to 14, wherein the organic stream comprises up to about 15% TSS.

16. The method according to any one of claims 8 to 15, wherein the SRT is between about 1.6 days to about 4.5 days.

17. The method according to any one of claims 8 to 16, wherein the HRT is between about 6 hours to about 18 hours.

18. The method according to any one of claims 8 to 17, wherein the VCF is between about 1.5 to about 2.2.
